(19) United States
(12) Patent Application Publication
(10) Pub. No.: US 2016/0280619 A1
(43) Pub. Date: Sep. 29, 2016

(54) PROCESS FOR THE PREPARATION OF 4-BROMO-1-CHLORO-2-(4-ETHOXYBENZYL)BENZENE

(71) Applicant: SUN PHARMACEUTICAL INDUSTRIES LIMITED, Mumbai, Maharashtra (IN)

(72) Inventors: Suresh Babu JAYACHANDRA, Hyderabad (IN); Devendra Prakash NAGDA, Udaipur (IN); Tarun Kumar SINGH, Lucknow (IN)

(21) Appl. No.: 15/033,088

(22) PCT Filed: Oct. 30, 2014
(86) PCT No.: PCT/IB2014/065726

§ 371 Date: Apr. 28, 2016

(30) Foreign Application Priority Data
Oct. 31, 2013 (IN) 3230/DEL/2013

(51) Int. Cl. C07C 41/30

(52) U.S. Cl. CPC C07C 41/30

(57) ABSTRACT

The present invention provides a process for the preparation of 4-bromo-1-chloro-2-(4-ethoxybenzyl)benzene of Formula III, which can be used as an intermediate for the preparation of dapagliflozin, or solvates thereof.

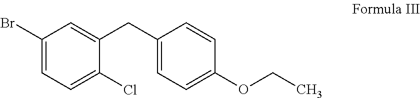 [SMILES: CCOc1ccc(Cc2cc(Br)ccc2Cl)cc1]

### FIELD OF THE INVENTION

The present invention provides a process for the preparation of 4-bromo-1-chloro-2-(4-ethoxybenzyl)benzene of Formula III, which can be used as an intermediate for the preparation of dapagliflozin, or solvates thereof.

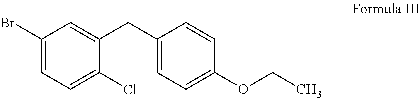 [SMILES: CCOc1ccc(Cc2cc(Br)ccc2Cl)cc1]

### BACKGROUND OF THE INVENTION

Dapagliflozin propanediol monohydrate is chemically designated as (1S)-1,5-anhydro -1 -C-[4-chloro-3-[(4-ethoxyphenyl)methyl]phenyl]-D-glucitol, (S)-propylene glycol, monohydrate and is marketed in Europe for the treatment of type 2 Diabetes mellitus. Its chemical structure is represented by Formula I:

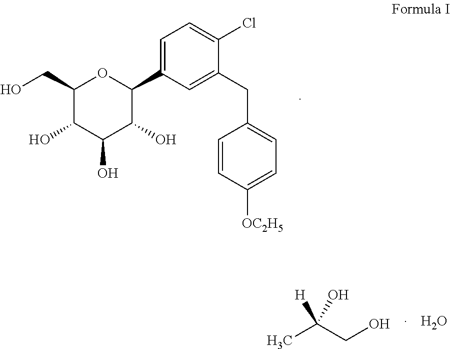

U.S. Pat. No. 6,515,117 (“the '117 patent”) discloses a process for the preparation of 4-bromo-1-chloro-2-(4-ethoxybenzyl)benzene of Formula III, comprising the reaction of 5-bromo-2-chlorobenzoyl chloride with phenetole thereby isolating 5-bromo-2-chloro-4′-ethoxybenzophenone, which upon reduction in acetonitrile at 50° C. gives 4-bromo-1-chloro-2-(4-ethoxybenzyl)benzene of Formula III. The '117 patent discloses that further increasing the temperature during the reduction step results in the formation of N-acetyl-5-bromo-2-chloro-4′-ethoxydiphenylmethylamine—an impurity of Formula VI. This impurity may be formed by the nucleophilic addition of acetonitrile to 5-bromo-2-chloro-4′-ethoxybenzophenone, followed by hydrolysis of the addition product.

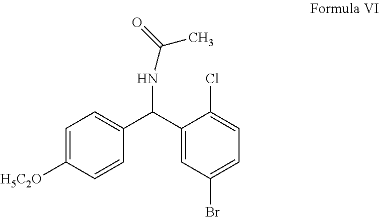

The present invention provides a one pot process for the preparation of 4-bromo-1-chloro-2-(4-ethoxybenzyl)benzene of Formula III that circumvents the use of acetonitrile as a solvent thus avoiding the formation of N-acetyl-5-bromo-2-chloro-4′-ethoxydiphenylmethylamine, an impurity of Formula VI.

### SUMMARY OF THE INVENTION

The present invention provides a process for the preparation of 4-bromo-1-chloro-2-(4-ethoxybenzyl)benzene of Formula III, which can be used as an intermediate for the preparation of dapagliflozin of Formula II, or solvates thereof.

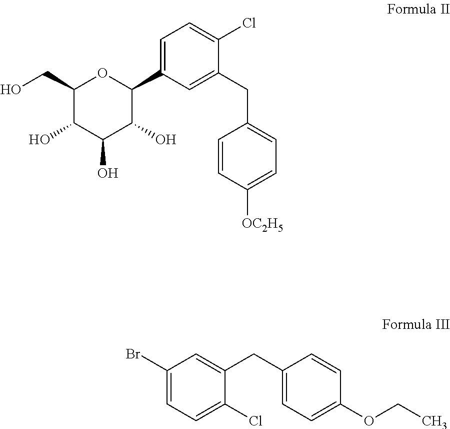 [SMILES: CCOc1ccc(Cc2cc([C@@H]3O[C@H](CO)[C@@H](O)[C@H](O)[C@H]3O)ccc2Cl)cc1 ; CCOc1ccc(Cc2cc(Br)ccc2Cl)cc1]

An aspect of the present invention provides a process for the preparation of a compound of Formula III,

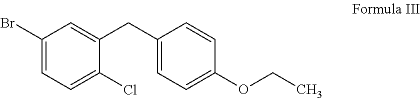 [SMILES: CCOc1ccc(Cc2cc(Br)ccc2Cl)cc1]

comprising reacting a compound of Formula IV with phenetole and reducing the reaction product thus obtained,

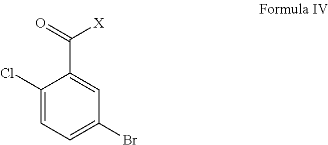

wherein ‘X’ is a leaving group and the reaction proceeds without isolating the reaction product obtained by the reaction of a compound of Formula IV with phenetole.

### DETAILED DESCRIPTION OF THE INVENTION

The term “about”, as used herein, refers to any value which lies within the range defined by a number up to ±10% of the value.

The term “substantially free of N-acetyl-5-bromo-2-chloro-4′-ethoxydiphenylmethylamine”, as used herein, refers to a compound having less than 1%, preferably less than 0.5%, and most preferably less than 0.1% of N-acetyl-5-bromo-2-chloro-4′-ethoxydiphenylmethylamine, an impurity of Formula VI. The term “substantially free of N-acetyl-5-bromo-2-chloro-4′-ethoxydiphenylmethylamine” includes a compound having no detectable amount of N-acetyl-5-bromo-2-chloro-4′-ethoxydiphenylmethylamine, an impurity of Formula VI.

The term “leaving group”, as used herein, refers to a halogen or an alkoxy group. Examples of halogens include fluorine, chlorine, bromine, and iodine. Examples of alkoxy groups include methoxy, ethoxy, propoxy, and butoxy.

In the context of the present invention, “solvates” refer to complexes of dapagliflozin with water, methanol, ethanol, n-propanol, propanediol, and butynediol.

The compound of Formula IV is prepared by reacting 5-bromo-2-chlorobenzoic acid with a reagent selected from the group consisting of thionyl chloride, phosphorus trichloride, phosphorus pentachloride, triphenylphosphine in carbontetrachloride, and cyanuric chloride in dimethylformamide.

In an embodiment of the present invention, the compound of Formula III is prepared by the reaction of a compound of Formula IV with phenetole in the presence of a Lewis acid followed by in situ reduction of the reaction product obtained.

In another embodiment of the present invention, the reduction is carried out in the presence of a solvent.

In another embodiment of the present invention, the reduction is carried out in the absence of acetonitrile.

In another embodiment of the present invention, the reaction of the compound of Formula IV with phenetole and the reduction of the reaction product formed proceeds without the isolation of a compound of Formula V.

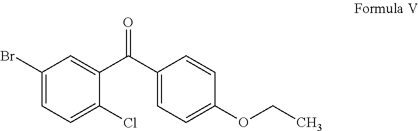 [SMILES: CCOc1ccc(C(=O)c2cc(Br)ccc2Cl)cc1]

Examples of Lewis acids include aluminum trichloride (AlCl₃), ferric chloride (FeCl₃), gallium trichloride (GaCl₃), boron trifluoride (BF₃), antimony pentachloride (SbCl₅), bismuth chloride (BiCl₃) and bismuth tris(trifluoromethanesulfonate) (Bi(OTf)₃).

The reducing agent, used for performing the reduction, is selected from the group consisting of metal hydrides and organosilanes. Examples of metal hydrides include lithium aluminum hydride, lithium diethoxyaluminum hydride, lithium triethoxyaluminum hydride, lithium tributoxyaluminum hydride, lithium dibutoxyaluminum hydride, lithium diethylaluminum hydride, lithium triethylaluminum hydride, K-selectride, L-selectride, diisobutylaluminum hydride, sodium borohydride, sodium cyanoborohydride, and tri-n-butyltin hydride. Examples of organosilanes include triethylsilane, tris(trimethylsilyl)silane, and diphenylsilane.

The solvent is selected from the group consisting of saturated hydrocarbons, halogenated hydrocarbons, ethers, polar organic solvents, or mixtures thereof. Examples of halogenated hydrocarbons include dichloromethane, carbon tetrachloride, and chloroform. Examples of saturated hydrocarbons include hexanes, heptanes, benzene, and toluene. Examples of ethers include diethylether, diisopropylether, tetrahydrofuran, and dioxane. Examples of polar organic solvents include dimethylformamide, N-methylpyridine, dimethylsulfoxide, and dimethylacetamide.

In another embodiment of the present invention, the compound of Formula III is substantially free of N-acetyl-5-bromo-2-chloro-4′-ethoxydiphenylmethylamine, an impurity of Formula VI.

In another embodiment of the present invention, the compound of Formula III is converted to dapagliflozin using the processes disclosed in our earlier filed applications (PCT/IB2014/064639, filed on Sep. 18, 2014, and PCT/IB2014/064676, filed on Sep. 19, 2014), the contents of both of which are incorporated herein by reference for their disclosure of the process of converting the compound of Formula III to dapagliflozin.

In yet other embodiment of the present invention, the dapagliflozin prepared using the compound of Formula III is substantially free of the impurity of Formula VI.

In general, 5-bromo-2-chlorobenzoic acid is reacted with oxalyl chloride to obtain a compound of Formula IV, which upon reaction with phenetole in the presence of aluminum chloride and reduction in the presence of sodium borohydride or triethylsilane gives the compound of Formula III. The synthesis of the compound of Formula III is carried out without the isolation of the intermediate of Formula V.

The conversion of the compound of Formula III into dapagliflozin can be carried out by following the processes described in U.S. Pat. Nos. 6,515,117, 7,375,213, 7,932,379, and 7,919,598, which are incorporated herein by reference.

### Methods:

The HPLC purity of dapagliflozin was determined using a Purospher® STAR RP-18e (150×4.6 mm), 3 μm column with a flow rate 1.0 to 1.5 mL/minute (flow gradient and organic gradient); column oven temperature: 25° C.; sample tray temperature: 25° C.; detector: UV at 225 nm; injection volume: 10 μL; run time: 60 min.

The following examples are set forth to aid the understanding of the invention but are not intended to and should not be construed to limit its scope in any way.

### EXAMPLES

### Example 1A

### Preparation of 4-Bromo-1-chloro-2-(4-ethoxybenzyl)benzene

Oxalyl chloride (0.8 mL) was added to a solution of 5-bromo-2-chlorobenzoic acid (2 g) in dichloromethane (20 mL) and dimethylformamide (0.2 mL) under a nitrogen atmosphere. The reaction mixture was stirred for one hour at 25° C. to 30° C. After completion of the reaction, the reaction mixture was concentrated under vacuum at 40° C. to 45° C. to obtain an oily residue. The oily residue was dissolved in dichloromethane (20 mL) and allowed to cool to 0° C. To this solution, phenetole (1.1 mL) and aluminum chloride (2.3 g) were added at 0° C. to 5° C. The reaction mixture was stirred at 0° C. to 5° C. for 2 hours. The reaction mixture was allowed to warm to a temperature of about 20° C. and triethylsilane (3.4 mL) was slowly added to it at the same temperature. The reaction mixture was stirred for about 36 hours at 20° C. to 25° C. After completion of the reaction, the reaction mixture was washed with an aqueous solution of sodium bicarbonate (8%; 20 mL). The layers were separated and the aqueous layer was extracted with dichloromethane (10 mL). The organic layers were combined and washed with water (20 mL). The organic layer was concentrated under vacuum to obtain 4-bromo-1-chloro-2-(4-ethoxybenzyl)benzene.

Yield: 2.5 g

### Example 1B

### Preparation of 4-Bromo-1-chloro-2-(4-ethoxybenzyl)benzene

Oxalyl chloride (0.8 mL) was added to a solution of 5-bromo-2-chlorobenzoic acid (2 g) in dichloromethane (20 mL) and dimethylformamide (0.2 mL) under an atmosphere of nitrogen. The reaction mixture was stirred for one hour at 25° C. to 30° C. After completion of the reaction, the reaction mixture was concentrated under vacuum at 40° C. to 45° C. to obtain an oily residue. The oily residue was dissolved in dichloromethane (2 mL) and allowed to cool to 0° C. To this solution, phenetole (1.1 mL) and aluminum chloride (1.25 g) were added at 0° C. to 5° C. The reaction mixture was stirred at 0° C. to 5° C. for one hour and tetrahydrofuran (20 mL) was added to the reaction mixture. Sodium borohydride (0.65 g) and aluminum chloride (2.3 g) were slowly added to the reaction mixture at 0° C. to 5° C. The reaction mixture was stirred for about 16 hours at 60° C. to 65° C. After completion of the reaction, the reaction mixture was concentrated at 50° C. to 55° C. and quenched with water (40 mL) at 0° C. to 15° C. The reaction mixture was extracted with toluene (2×20 mL). The organic layer was washed with water (10 mL) and concentrated under vacuum to obtain an oily residue. The residue was again dissolved in ethanol (10 mL) and concentrated under vacuum at 50° C. to 55° C. to obtain an oily residue. The residue was then dissolved in ethanol (10 mL) and stirred for 2 hours at −20° C. to −15° C. to obtain a solid. The solid was filtered, washed with pre-cooled ethanol (2 mL), and dried under vacuum at 25° C. to 30° C. for 3 hours to obtain 4-bromo-1-chloro-2-(4-ethoxybenzyl)benzene.

Yield: 1.2 g; HPLC: 95.21%

We claim:

1. A process for the preparation of a compound of Formula III, 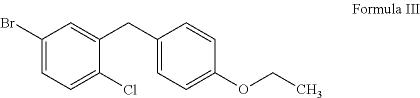 [SMILES: CCOc1ccc(Cc2cc(Br)ccc2Cl)cc1]
comprising reacting a compound of Formula IV with phenetole and reducing the reaction product thus obtained, 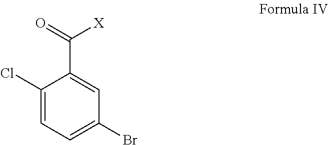
wherein ‘X’ is a leaving group and the reaction proceeds without isolating the reaction product obtained by the reaction of a compound of Formula IV with phenetole.

2. The process according to claim 1, wherein the process is carried out in the presence of a Lewis acid selected from the group consisting of AlCl₃, FeCl₃, GaCl₃, BF₃, SbCl₅, BiCl₃, and Bi(OTf)₃.

3. The process according to claim 1, wherein the reduction is carried out in the presence of a reducing agent selected from the group consisting of metal hydrides and organosilanes.

4. The process according to claim 1, wherein the process is carried out in the absence of acetonitrile.
